(19) United States
(12) Patent Application Publication
(10) Pub. No.: US 2002/0069958 A1
(43) Pub. Date: Jun. 13, 2002

(54) Application devices

(76) Inventors: John Vincent Fontana, Cincinnati, OH (US); Matthew Howard Wasson, Cincinnati, OH (US); Kenneth Edward Lamb, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/054,113

(22) Filed: Oct. 20, 2001

Related U.S. Application Data
(60) Provisional application No. 60/255,045, filed on Dec. 12, 2000.

(51) Int. Cl. A61F 13/00

(52) U.S. Cl. 156/263; 602/41

(57) ABSTRACT

Application devices comprising an applicator substrate, an interposed patch, and a release substrate. The application substrate and release substrate have graspable tabs allowing the user to grasp the tab of the applicator substrate, to grasp the tab on the release substrate, and to apply the patch to a target surface without releasing the tabs as originally grasped.

### CROSS REFERENCE

This application claims priority under Title 35, United States Code 119(e) from Provisional Application Serial No. 60/255,045, filed Dec. 12, 2000.

### TECHNICAL FIELD

The present invention relates to an application device for applying an adhesive patch.

### BACKGROUND

While many application devices for adhesive patches have been described, various problems are associated with their use. See, for example, U.S. Pat. Nos. 4,913,138; 5,106,629; 5,397,297; 5,511,689; 5,656,282; 5,685,833; 5,891,078; 6,129,929; and international publication number WO 98/17216. Use of many of these devices, for example in the application of an adhesive patch, results in the patch curling or adhering to itself before it can be secured to the target surface. Another problem with many application devices is that the user is forced to touch the surface of the patch during application. This is particularly problematic when the patch is intended to be sterile or contains an adhesive surface. Such contact either contaminates the patch or reduces the adhesiveness of the patch.

There are one-handed patch application devices in the art that reportedly minimize the occurrence of a user having to touch the surface of the patch. However, many of these devices have a tab on the applicating member that overlaps the releasing member. Such a design makes it difficult for the user to grasp the tab and pull the applicating member away from the releasing member. Still other one-handed patch application devices require the user to grasp the applicating member in one location, to release the applicating member from the releasing member, and then to grasp the applicating member in another location to apply the patch to the target surface. Still other devices have adhesive disposed throughout the applicating member, thereby diffusing actives, dyes, etc., from the patch into the adhesive and diminishing the effectiveness of the patch. Still other devices have a limited number of adhesives that can be utilized and function as a one-handed device. Still other devices pose narrow manufacturing tolerance ranges during assembly.

For the foregoing reasons, there is a continuing need for a patch application device that: applies a patch to a target surface without the patch curling upon itself; without the patch unnecessarily being touched by the user; that allows a user to easily remove the releasing member from the applicating member containing the patch, in turn, applying the patch to a target surface and thereby removing the applicating member with the patch affixed to the target surface; without the user releasing the applicating member as originally grasped; with minimal diffusion of an active of the patch diffusing into the adhesive disposed on the applicating member; with diverse number of operable adhesives and materials; and with increased manufacturing tolerances.

### SUMMARY OF THE INVENTION

The present invention provides an application device that comprises an applicator substrate, a release substrate, and a patch interposed therebetween suitable to apply to a target surface. The applicator substrate is substantially planar and has an outwardly extending graspable tab. The applicator substrate has an interior surface that has an adhesive means that comprises an adhesive area. The patch is substantially planar and has a first and second surface. The adhesive area of the applicator substrate releasably affixes the first surface of the patch thereby comprising a first peel bond and thereby forming an applicator substrate/patch combination (hereinafter “combination”). The combination has an upper surface. The second surface of the patch comprises an adhesive means. The release substrate is substantially planar, has an outwardly extending graspable tab, and has an interior surface. The interior surface of the release substrate is releasably affixed to the upper surface of the combination thereby comprising a second peel bond. Further, the tab of the releasably affixed release substrate is offset laterally from the combination. The strength of the first peel bond is greater than the strength of the second peel bond thus enabling the user to separate the release substrate from the combination. Upon removing the release substrate and applying the upper surface of the combination to a target surface, an adhesive bond is formed between the second surface of the patch and the target surface. The strength of the adhesive bond is greater than the strength of the first peel bond thereby enabling the applicator substrate to be removed from the patch after applying the patch to the target surface.

### BRIEF DESCRIPTION OF THE DRAWINGS

While the specification concludes with claims particularly pointing out and distinctly claiming the invention, it is believed that the present invention will be better understood from the following description taken in conjunction with the accompanying drawings in which:

FIG. 1 is an exploded view of a first embodiment of the application device made in accordance with the present invention;

FIG. 2 is a top plan view of the of the first embodiment of the invention prior to removal of the release substrate;

FIG. 3 is a top plan view of a second embodiment of the invention after removal of the release substrate;

FIG. 4 is a schematic illustration of a process suitable for manufacturing the first embodiment of the invention.

### DETAILED DESCRIPTION OF THE INVENTION

Reference will now be made in detail to embodiments of the invention, examples of which are illustrated in the accompanying drawings wherein like numerals indicate the same elements throughout the views and wherein elements having the same two last digits (e.g., 20 and 120) or an alphabetic suffix (e.g., 22A and 22B) connote similar elements.

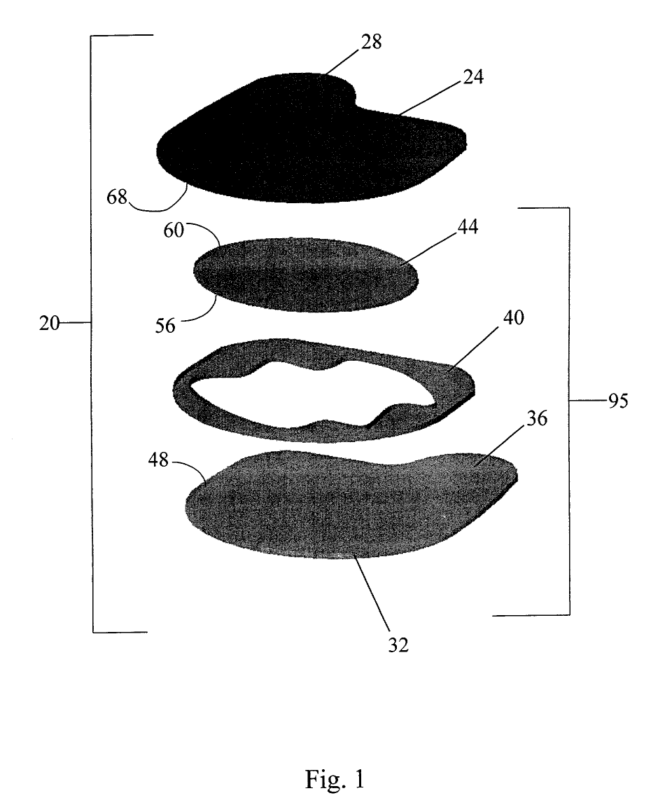

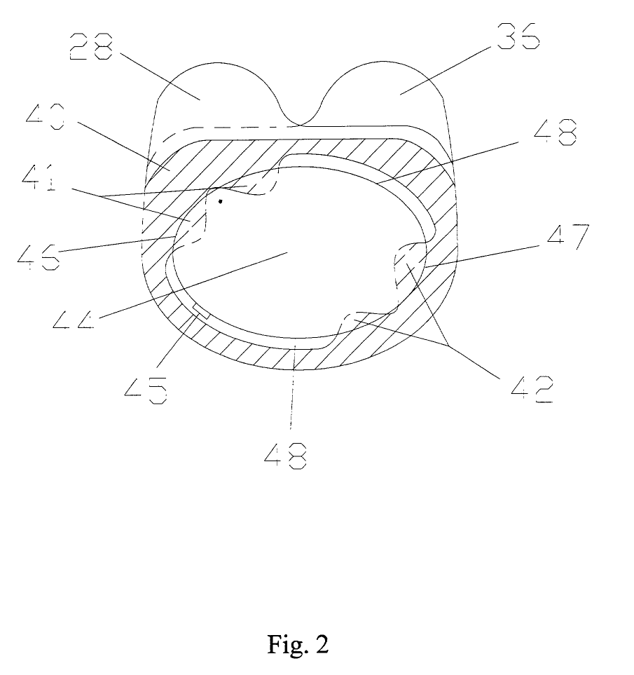

FIGS. 1 and 2 illustrate a first embodiment of the invention. FIG. 1 is an exploded view while FIG. 2 is a top plan view. A laminar application device 20 of the present invention comprises a substantially planar applicator substrate 32, an interposed substantially planar patch 44, and a substantially planar release substrate 24. The applicator substrate 32 comprises a graspable tab 36 extending outwardly therefrom. The applicator substrate 32 also comprises an interior surface 48. The interior surface 48 of the applicator substrate 32 comprises an adhesive means that is bonded, coated, formed, laminated, or otherwise applied to the interior surface 48 of the applicator substrate 32 to comprise an adhesive area 40. The adhesive area 40 shown in FIGS. 1 and 2 extends about 40% of the interior surface 48 but does not extend to the tab 36 of the applicator substrate 32. Preferably the adhesive area extends about 1% to about 95%, more preferably about 20% to about 60%.

Still referring to FIGS. 1 and 2, the device 20 further comprises a substantially planar patch 44 interposed between the applicator substrate 32 and release substrate 24. The patch 44 comprises a first surface 56 and a second surface 60. The adhesive area 40 partially underlies and releasably adheres the patch 44 to the interior surface 36 of the applicator substrate 32. The adhesive area 40 also releasably adheres the release substrate 24. The first surface 56 of the patch 44 is releasably affixed to the interior surface 48 of the applicator substrate 32 by the adhesive means (having an adhesive area 40), thereby comprising a first peel bond, and thereby forming an applicator substrate/patch combination 95 (hereinafter “combination”). As used herein, “peel bond” means a bond formed between two surfaces adhered to each other as measured by a bond force. As used herein, “bond force,” means the amount of force necessary to separate the two surfaces from each other thereby measuring the strength of the bond. Thus, if the strength of a first peel bond is greater than the strength of a second peel bond, a greater bond force is necessary to separate the surfaces comprising the first bond as compared to the surfaces comprising the second peel bond.

As shown in FIG. 2, the adhesive area 40 that adheres the first surface 56 of the patch 44 comprises an adhesive contact area having a leading edge contact area 41 and trailing edge contact area 42. Preferably the releasably affixed patch 44 does not overlap the tab 36 of the applicator substrate 32. The combination 95 comprises an upper surface (not shown).

As shown in FIG. 1, the second surface 60 of the patch 44 comprises an adhesive means (not shown) that is also bonded, coated, formed, laminated, or otherwise applied to the second surface 60 of the patch 44. The adhesive means of the second surface 60 of the patch 44 is preferably continuous with the second surface 60 of the patch 44. The adhesive means of the second surface 60 of the patch 44 is exposed on the upper surface of the combination 95. Although not shown in FIG. 1, when a user applies the upper surface of the combination 95 to a target surface, the adhesive means of the second surface 60 of the patch 44 affixes the patch 44 to the target surface thereby comprising an adhesive bond. The strength of an adhesive bond is measured by a bond force.

Referring to FIGS. 1 and 2, the device 20 includes a release substrate 24 comprising a graspable tab 28 extending outwardly therefrom. The release substrate 24 comprises an interior surface 68 that is releasably affixed to the upper surface of the combination 95 thereby comprising a second peel bond. The adhesive means of the second surface 60 the patch 44, or the adhesive means of the interior surface 48 of the applicator substrate 32, or a combination thereof, releasably adheres the release substrate 24 to the combination 95. Preferably when the release substrate 24 is releasably affixed to the combination 95, the interposed patch 44 is enclosed between the applicator substrate 32 and the release substrate 24 thereby comprising a closed container for the patch 44.

Referring to FIGS. 1 and 2, when assembled as a device 20, the outwardly extending tab 28 of the release substrate 24 is offset laterally from the combination 95 to allow for easy grasping by the user. As used herein, “offset laterally” means the tab 28 of the releasably affixed release substrate 24 is at least partially offset laterally from the combination 95 that includes the tab 36 of the applicator substrate 32. In one embodiment, the tab 28 of the release substrate 24 is completely offset laterally from the combination 95. As shown, the graspable tabs 36, 28 of the release substrate 24 and the applicator substrate 32 are laterally offset from each other and are not bonded together. The respective tabs 36, 28 are arranged such that the user can grasp the respective tabs 36, 28 simultaneously without having to digitally separate them. As shown, both tabs 36, 28 are free for grasping.

To facilitate the use of the device 20, the tabs 36, 28 of the applicator substrate 32 and the release substrate 24 may be identified and distinguished by a variety of means including color, printing, labeling, varying tab shape/size, transparity/opaqueness of the materials, or any combination thereof. The device 20 may be accompanied by instructions detailing how the device 20 is to be applied.

Applicants discovered that a significant factor in determining the strength of the first peel bond is the pattern of the adhesive area 40, more specifically the adhesive contact area. Referring to FIGS. 1 and 2, the adhesive contact area has a leading edge contact area 41 and a trailing edge contact area 42. The patch 44, when releasably affixed to the applicator substrate 32 thereby forming the combination 95, comprises a circumferential lateral edge 45. As used herein, the “circumferential lateral edge” means the lateral edge of the outer most surrounding boundary of the patch 44. The circumferential lateral edge 45 has a leading edge 46, a trailing edge 47, and a non-securing edge 48. The leading edge 46 is proximate to the tab of the release substrate 28 when the release substrate 24 is releasably affixed to the combination 95. The tab 28 of the release substrate 24 dictates the peel direction of the release substrate 24 as the user removes the release substrate 24 from the combination 95. As used herein, “peel direction” means the course taken by one laminate as it is peeled from another laminate. The leading edge 46 of the patch 44 of the combination 95 is the first exposed surface of the patch 44 as the user removes the release substrate 24 from the combination 95. The trailing edge 47 is distal to the tab 28 of the release substrate 24. As the user continues to remove the release substrate 24 from the combination 95, the trailing edge 47 is the last exposed surface of the patch 44.

The leading edge contact area 41 tacks the leading edge of the patch 46 to the interior surface 48 of the applicator substrate 32 when the release substrate 24 is removed from the combination 95. Thus, the leading edge of the patch 46 is the part of the circumferential edge 45 of the patch 44 that is tacked by the adhesive contact area, and that is first exposed as a user removes the release substrate 24 from the combination. The leading edge 46 need not be continuous with the leading edge contact area 41 (as shown in the first embodiment of the present invention). Preferably the leading edge 46 comprises about 1% to about 45%, more perferably about 10% to about 30% of the total circumferential lateral edge 45 of the patch 44.

Similarly, the trailing edge contact area 42 tacks the trailing edge 47 to the interior surface 48 of the applicator substrate 32 when the release substrate 24 is removed from the combination 95. The leading edge 47 need not be continuous with the trailing edge contact area 42 (as shown in the first embodiment of the present invention). Preferably the trailing edge 47 comprises preferably about 1% to about 50%, more preferably about 10% to about 35% of the total circumferential lateral edge 45 of the patch 44. In the first embodiment of the invention, the leading edge 46 of the patch is diagonal to the trailing edge 47 of the patch. Similarly, in the first embodiment of the invention, the leading edge contact area 41 is diagonal to the trailing edge contact area 42.

The non-securing edge 48 is the part of the circumferential lateral edge 45 of the patch 44 that is not tacked by the adhesive contact area. Preferably the non-securing edge comprises about 5% to about 95%, more preferably about 40% to about 80%, of the total circumferential lateral edge 45 of the patch 44.

By tacking the leading edge 46 and the trailing edge 47, the adhesive contact area facilitates a patch 44 being applied to a target surface without the patch 44 curling upon itself and without the user unnecessarily touching the patch 44. Preferably the leading edge contact area 46 comprises an area of 0.1 cm² to 4000 cm², more preferably 0.4 cm² to 2 cm². Preferably the trailing edge contact area 42 comprises an area of 1 cm² to 4500 cm², more preferably 0.4 cm² to 2.5 cm².

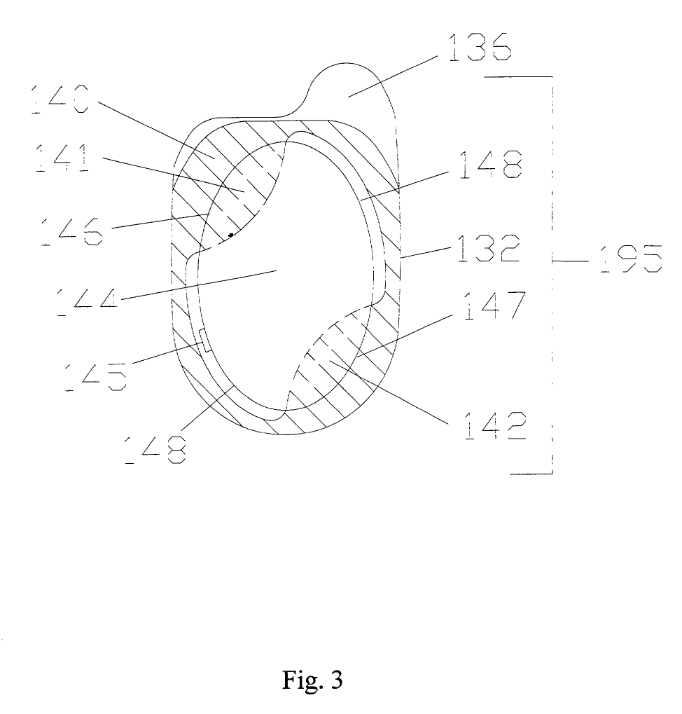

FIG. 3 is a top plan view of the combination 195 of a second embodiment of the invention after removal of the release substrate (not shown in FIG. 3). The combination 195 comprises a patch 144 releasably affixed to an applicator substrate 132, wherein the applicator substrate 132 comprises a graspable tab 136 extending outwardly therefrom. The applicator substrate 132 comprises an adhesive means that is bonded to the applicator substrate to comprise an adhesive area 140. The patch is releasably affixed to the adhesive area 140 of the applicator substrate 132. The adhesive area 140 that adheres the patch 144 comprises an adhesive contact area that has a leading edge contact area 141 and a trailing edge contact area 142 that are each of a curvilinear “scallop-shaped” pattern. The patch 144, in turn, comprises a circumferential lateral edge 145 having a leading edge 146, a trailing edge 147, and a non-securing edge 148. Although not shown in FIG. 3, the leading edge 146 is proximate the tab of the release substrate when the release substrate is releasably affixed to the combination 195.

Referring to FIG. 1, the strength of the first peel bond is greater than the strength of the second peel bond such that when the user removes the release substrate 24 from the combination 95, the patch 44 remains releasably affixed to the applicator substrate 32. Further, the strength of the adhesive bond is greater than the strength of the first peel bond such that when the user applies the upper surface of the combination 95 to the target surface, the patch 44 affixes to the target surface and releases from the combination 95. To this end, the present invention provides increased bond strength ratios between the three bonds thereby enhancing the performance of the device; providing a greater range of operable adhesive types; providing a greater range of operable patch sizes; providing greater tolerance to design modifications; and requiring less overall adhesive to be utilized. In turn, utilizing less adhesive provides for savings in manufacturing costs, diminishing the amount of active that is able to diffuse from the patch 44 to the adhesive of the interior surface 48 of the applicator substrate 32, and diminishing the amount of Pextractables from the adhesive of the interior surface 48 to the patch 44.

Referring to FIGS. 1 and 2, Applicants also discovered that if the leading edge contact area 41 and the trailing edge contact area 42 are patterned in a sinusoidal manner as illustrated by the first embodiment of the invention, greater tolerance for manufacturing variations are exhibited. As used herein, “manufacturing variations” means those variations that occur during the many steps of mass producing the device of the present invention such as, but not limited to: the placement of the patch 44 onto the adhesive area 40. For example, with many adhesive area 40 patterns, the patch 44 must be applied precisely to adhesive area 40 during manufacturing. By patterning the leading edge contact area 41 and trailing edge contact area 42 in a sinusoidal manner, Applicants observed that the patch 44 can be applied to the applicator substrate 32 during manufacturing with increased tolerances. For example, Applicants observed an embodiment of the device that represented a patch placement area of 122% as compared to the area of the patch. More specifically, in the embodiment so described, the interior surface of the applicator substrate, not including the area of the tab, is 32.5 cm², the first surface of the patch is 18 cm² area, and a patch placement area 22 cm².

Referring to FIG. 2, the sinusoidal pattern of the adhesive contact area may have a number of curves. Furthermore, these curves need not be symmetrical with one another.

Still referring to FIG. 2, Applicants observed that theoretical peel force (Newtons) of the first peel bond can be predicted by multiplying the maximum contact width (cm) by an adhesive's art-accepted adhesive peel force coefficient (Newtons/cm). As used herein, “maximum contact width” means the greatest linear dimension perpendicular to the peel direction within the leading edge contact area 41 or trailing edge contact area 42. Actual peel force can be measured by a compression/tensile tester (Instron), or a Friction/Peel Tester (Thwing Albert).

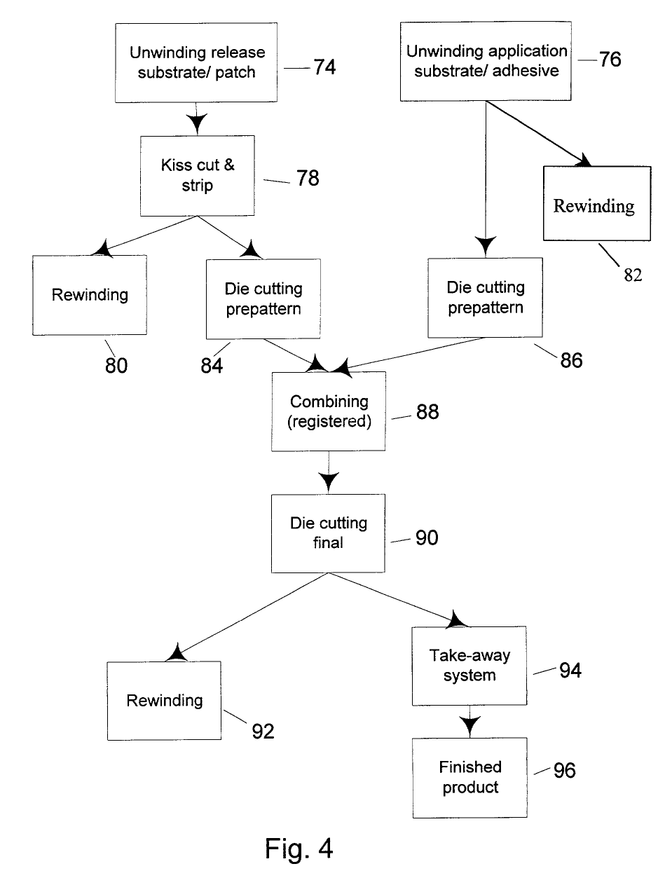

FIG. 4 is a schematic illustration for a process suitable for manufacturing the application devices herein. The transformation flow chart generally represents two paths that converge and combine to form the final application device product. The manufacturer first combines overlapping webs of a release substrate web and a patch web into a stock roll (without limitation, the manufacturer is Watson Laboratories, Inc., Salt Lake City, Utah). The manufacturer also first combines overlapping webs of an applicator substrate web, an adhesive web that has been cut in the desired pattern, and an adhesive web release liner into a stock roll (without limitation, the manufacturer is Marion, Inc., Indianapolis, Ind.). The first path of the transformation flow chart begins with the unwinding of stock release substrate web and patch web 74. The unwinding process consists of converting these stock materials that are in rolls, into continuous flat sheets. The second path of the transformation flow chart begins with the unwinding of stock applicator substrate web, adhesive web, and adhesive web release liner 76. As with the first path, the unwinding process of the second path consists of converting these stock materials that are in rolls, into continuous flat sheets while the adhesive web release liner is removed and rewound 82. Returning to the first path, the combined release substrate web and patch web are “kiss cut” 78, i.e. cutting the patch film without cutting the release liner web. Concomitantly, scrap patch film is stripped off for rewinding 80. In the next steps of both the first and second pathways, the tabs of the respective release substrate web and application substrate web are die cut to slit the tab portions of the respective webs 84, 86 without removing or dislodging any material. The first and second pathways converge in a combining step 88 wherein the applicator substrate web and release substrate web are combined in a registered fashion. The subsequent die cutting-final step 90 die cuts the combined webs to the desired finished shape and appropriately spaces the finished application devices as they move along on a conveyor. The final die cutting step also removes scrap material. To this end, a final rewinding step 92 rewinds this scrap material. As to the final application devices, a take-away system 94 accepts incoming individual application devices off of the conveyor and places them in a collection unit where finished product is packaged 96. An optional packaging step is not shown, but can be included according to the desires of the manufacturer.

The device of the present invention minimizes user confusion and facilitates ease of use. Many other devices require the user to grasp the applicating member in one location to release the applicating member from the releasing member, and then to grasp the applicating member in another location to apply the patch to the target surface. In contrast, and referring to FIGS. 1 and 2, a user of the present invention grasps the tab 36 of the applicator substrate 32 and retains her grasp on the tab 36 during the entire process of using the device 20 to deliver a patch 44 to a target surface. In an embodiment, the user retains the grips on both the tab 36 of the applicator substrate 32 and the tab 28 of the release substrate 24 during the entire process of the using the device 20 to deliver the patch 44 to the target surface. In another embodiment, the user retains the grasp on the tab 36 of the applicator substrate 32 during the entire process of the using the device 20 and presses on the exterior side of the combination (not shown) with the other hand after applying the combination 95 to the target surface and before removing the applicator substrate 32, thereby affixing the patch 44 to the target surface.

Other factors that affect bond strength, and hence bond force, include, but are not limited to: amount and type of adhesive, type of materials used and flexibility thereof, the size and thickness of each laminate, and the amount of force used to combine the laminates when constructing the device.

Suitable adhesives for use as either of the adhesive means are intended in the broadest sense. Non limiting examples of adhesives include: hook and loop closures, resin adhesives, latex; hotmelts, thermoplastic adhesives, acrylics, vinyl acetates, natural and synthetic rubbers, natural and synthetic gums, polysiloxanes, polyacrylates, ethylene/vinyl acetate copolymers, polyvinylpyrrolidones, vinylpyrrolidone copolymers and particularly vinyl pyrrolidone/vinylacetates, styrene block copolymers, natural or synthetic polysaccharides, cellulose materials, and mixtures therefrom.

In one embodiment, the adhesive means is a low tactile adhesive that is bonded to the first surface of the applicator substrate so that when the patch is removed from the applicator substrate, substantially all the first adhesive means remains on the applicator substrate. Non limiting examples of low tactile adhesives include National Starch Acrylic adhesive #9296.

In another embodiment, the adhesive means is a double-sided high tack/low tack adhesive tape wherein the high tack side is bonded to the interior surface of the applicator substrate. Non limiting examples of high tack/low tack adhesives include those from 3M sold under the product description “9415” and “9416.” In yet another embodiment, the adhesive means is a double-sided high tack/medium tack adhesive tape wherein the high tack side is bonded to the interior surface of the applicator substrate. A non limiting example of a double-sided high tack/medium tack adhesive tape includes one available from 3M under the product description “9425.” In still yet another embodiment, the adhesive means is a low tactile acrylic adhesive that is bonded the second surface of the patch.

Suitable materials for use as the applicator substrate, patch, and release substrate are intended in their broadest sense. Non limiting examples include polyethylene, polyester, polypropylene, polyurethane, polyolefin, polyvinyl alcohol, polyvinyl chloride, polyvinylidene, polyamide, vinyl acetate resins, ethylene/vinyl acetate copolymers, ethylene/ethylacrylate copolymers, metal-vapor deposited films or sheets thereof, rubber sheets or films, expanded synthetic resin sheets or films, non-woven fabrics, fabrics, knitted fabrics, foils and papers. These materials can be used singularly or in combination, as laminates or as coextrusions, to form the applicator substrate, patch, and release substrate. The release liner may also be coated with a coating material such as silicone, TEFLON™, or thermoplastic materials such as polyester, polyvinyl resin, polyethylene or cellulose acetate, by any conventional means. By example only, different materials, production methods, and specifications are described in Hanlon, Joseph F., Handbook of Package Engineering, Technomic Publishing Co. (1992), incorporated herein by reference.

In one embodiment, the applicator substrate is polyester film. In another embodiment the patch is polyethylene film. In yet another embodiment, the release substrate is siliconized polyester.

Flexibility affects a peel bond. The type of material and the thickness thereof, in turn, determines flexibility. Preferably the applicator substrate has a thickness of 0.002 cm to 0.1 cm, more preferably 0.005 cm to 0.02 cm. Preferably the patch has a thickness of 0.002 cm to 0.05 cm, more preferably 0.005 cm to 0.013 cm. Preferably the release substrate has a thickness of 0.002 cm to 0.1 cm, more preferably 0.005 cm to 0.013 cm. In one embodiment, the Release Substrate is more flexible than Application Substrate. Flexibility can be measured by a compression/tensile tester (Instron).

The size of each laminate of the device may vary. Preferably the applicator substrate has an area of 1 cm² to 5000 cm², more preferably 25 cm² to 40 cm². Preferably the adhesive area has an area of 0.5 cm² to 4800 cm², more preferably 5 cm² to 20 cm². Preferably the patch has an area of 0.5 cm² to 4800 cm², more preferably 5 cm² to 28 cm². Preferably the release substrate has an area of 1 cm² to 5000 cm², more preferably 25 cm² to 40 cm².

The amount of force used to combine the laminates when constructing the present device 20 affects peel force. Preferably the combining force is from about 1 KPa to 175 KPa, more preferably about 5 KPa to about 40 KPa.

The patch of present invention can be applied to a target surface for many purposes. For example, a patch comprising latex may be applied to a puncture to fix a flat tire. The patch would seal the puncture and prevent air from escaping. A sterile patch comprising gauze may be applied to a skin wound to stop bleeding or to promote healing. A patch containing graphics could be applied to a surface to apply a sticker. A 1 or 2 sided tape could be applied to a surface using the application substrate. A large area patch containing graphics, printing or clear could be applied like “shelf paper” using the application substrate. A patch comprising peroxide may be applied to teeth as a whitening strip. Some other examples include applying a patch to a target surface that is in need for reinforcement, protection from abrasions, protective padding, or cushioned absorbent.

In another example, the patch is applied to the skin for cosmetic or medicinal purposes. In such an example, the patch comprises a releasable cosmetic or medicinal ingredient. Such ingredients, hereinafter referenced to as “actives,” can be water-soluble, oil-soluble, or soluble in both water and in oil. Included among such ingredients are antimicrobials, rubifacients, analgesics, hormones, anti-seborrhea agents, anti-psoriatic agents, keratolytic agents, and the like. Such actives can be selected from a wide variety of such materials that are known to be useful when affixed topically. Reference can be made to the United States Pharmacopeia for lists of such actives. A safe and effective amount of active is contemplated. A “safe and effective amount” of active is an amount that is effective as a cosmetic or medicinal ingredient without undue adverse side effects (such as toxicity, irritation, or allergic response), commensurate with a reasonable benefit/risk ratio when used in the manner of this invention.

When the patch is intended to deliver an active such as a drug, the patch may also contain agents known to accelerate the delivery of a drug through the skin or mucosa. These agents have been referred to as skin-penetration enhancers, accelerants, adjuvants, and sorption promoters, and are herein referred to collectively as “enhancers.”

In addition to enhancers, there may also be incorporated various pharmaceutically acceptable additives and excipients available to those skilled in the art. These additives include tackifying agents such as aliphatic and aromatic hydrocarbons; binders such a lecithin; rheological agents such as fumed silica; as well as diluents, stabilizers, filler, clays, buffering agents, biocides, humectants, antiirritants, antioxidants, preservatives, colorants, and the like.

### EXAMPLE 1

An application device of the present invention, as illustrated in FIGS. 1 and 2, is made by combing the laminates as follow:

Firstly, an applicator substrate is die cut from a sheet of polyester film purchased from Marion, Inc., under the product description “Type-D Mylar” of a thickness of 0.127 mm. The applicator substrate has an overall width of 6 cm and a length of 6.5 cm (including the tab) and has an overall area of 33 cm² wherein the tab has an area of 3 cm².

Secondly, the adhesive means of the interior surface of the applicator substrate is a double-sided tape high tack/low tack adhesive purchased from 3M Converter Markets, under the product description of “9596” with the high tack side bonded to the interior surface of the applicator substrate. The adhesive area is subsequently kiss-cut such that the leading contact area and the trailing edge contact area are of a sinusoidal pattern as illustrated in FIG. 2. The adhesive area is 13 cm². The leading edge contact area is 0.7 cm² and the trailing edge contact area is 0.8 cm².

Thirdly, the patch is die cut from a sheet of polyethylene film purchased from 3M under the product description “#9720” of 0.008 cm thickness. However, before being die cut, the adhesive means of the second surface of the patch is coated on to the sheet. The adhesive means is an acrylic adhesive purchased from National Starch and Adhesives under the product description “Dura-Tak 87-2888” such that the adhesive is continuous with the sheet. The patch is die cut from the sheet with the coated acrylic adhesive such that the patch is an oval shape with a width of 3.5 cm and a length of 5 cm with an overall area of 14 cm². The patch is placed upon the adhesive area such that leading edge is 20%, the trailing edge is 21%, and the non-securing edge is 59% of the circumferential lateral edge.

Lastly, a release substrate is die cut from a sheet of siliconized polyester purchased from Loparex under the product description as “3 mil CL PET EXP 1000 EXP=X6AA12 HZ.” of a thickness of 0.008 cm. The release substrate has an overall width of 6 cm and a length of 6.5 cm (including the tab) and has an overall area of 33 cm² wherein the tab has an area of 3 cm².

The laminates of the device are combined with a force of 7 KPa at a manufacturing speed of 120 patches/min.

The peel force of the first peel bond is 0.056N; the peel force of the second peel bond is 0.007N; and the peel force of the adhesive bond is 0.088N.

The patch of the foregoing type can be used to deliver hormones as part of a hormone therapy to a human female patient in need thereof.

### EXAMPLE 2

A kit for applying a hormone patch according to the present invention is packaged in a singe-source container, as follows:

### Kit

An application device according to Example 1.

A safe and effective amount comprising 14.2 mg coated of testosterone is coated onto the patch per conventional means.

Instructions for Use.

A user in need of a hormone patch, per the instructions of the kit, applies the patch comprising testosterone utilizing the application device.

While particular embodiments of the present invention have been illustrated and described, it will be clear to those skilled in the art that various changes and modifications may be made without departing from the spirit and scope of the invention, and it is intended to cover in the appended claims all such modifications that are within the scope of the invention.

What is claimed is:

1. A laminar application device for applying an interposed patch to a target surface, comprising:
a substantially planar applicator substrate comprising a graspable tab extending outwardly therefrom; wherein the applicator substrate comprises an interior surface the substantially planar patch comprising a first surface and a second surface and a substantially planar release substrate comprising a graspable tab extending outwardly therefrom; wherein the release substrate comprises an interior surface;
wherein the interior surface of the applicator substrate comprises an adhesive means;
wherein the second surface of the patch comprises an adhesive means;
wherein the adhesive means of the interior surface of the applicator substrate releasably affixes the first surface of the patch to the interior surface of the patch thereby comprising a first peel bond and thereby forming an applicator substrate/patch combination comprising an interior surface;
wherein the release substrate is releasably affixed to the upper surface of the combination thereby comprising a second peel bond and wherein the tab of the release substrate is offset laterally from the combination;
wherein the strength of the first peel bond is greater than the strength of the second peel bond strength; and
wherein the strength of an adhesive bond between the adhesive means of the second surface of the patch and a target surface is greater than the strength of the first peel bond.

2. A device according to claim 1, wherein the adhesive means of the interior surface of the applicator substrate further comprises an adhesive area comprising an adhesive contact area;
wherein the adhesive contact area comprises a leading edge contact area;
wherein the patch comprises a circumferential lateral edge; wherein the circumferential lateral edge comprises a leading edge; and a non-secured edge;
wherein the leading edge is proximate to the tab of the releasably affixed release substrate; and
wherein the leading edge contact area tacks the leading edge to the interior surface of the applicator substrate.

3. A device according to claim 2, wherein the adhesive contact area further comprises a trailing edge contact area;
wherein the circumferential lateral edge further comprises a trailing edge;
wherein the trailing edge is distal to the tab of the releasably affixed release substrate;
wherein the trailing edge contact area tacks the trailing edge to the interior surface of the applicator substrate.

4. A laminar application device for applying an interposed patch to a target surface, comprising:
a substantially planar applicator substrate comprising a graspable means; wherein the applicator substrate comprises an interior surface; the substantially planar patch comprising a first surface and a second surface; and a substantially planar release substrate comprising a graspable means; wherein the release substrate comprises an interior surface;
wherein the interior surface of the applicator substrate comprises an adhesive means;
wherein the second surface of the patch comprising an adhesive means;
wherein the adhesive means of the interior surface of the applicator substrate releasably affixes the first surface of the patch thereby comprising a first peel bond and thereby forming an applicator substrate/patch combination comprising an interior surface;
wherein the release substrate is releasably affixed to the interior surface of the combination thereby comprising a second peel bond;
wherein the adhesive means of the interior surface of the applicator substrate comprises an adhesive area comprising an adhesive contact area;
wherein the adhesive contact area has a leading edge contact area and a trailing edge contact area;
wherein the patch comprises a circumferential lateral edge wherein the circumferential lateral edge has a leading edge a trailing edge; and a non-securing edge;
wherein the leading edge is proximate to the graspable means of the releasably affixed release substrate;
wherein the trailing edge is distal to the graspable means of the releasably affixed release substrate;
wherein the leading edge contact area tacks the leading edge to the interior surface of the applicator substrate;
wherein the trailing edge contact area tacks the trailing edge to the interior surface of the applicator substrate; and
wherein the strength of the first peel bond is greater than the strength of the second peel bond.

5. A device according to claim 4 wherein the strength of an adhesive bond between the adhesive means of the second surface of the patch and a target surface is greater than the strength of the first peel bond.

6. A device according to claim 5 wherein the graspable means of the applicator substrate and the release substrate are outwardly extending tabs and wherein the tab of the release substrate is laterally offset from the combination.

7. A device according to claim 6, wherein at least either the leading edge contact area or the trailing edge contact area is a sinusoidal pattern.

8. A device according to claim 7, wherein the adhesive area comprises surrounding the patch.

9. A device according to claim 8, wherein the patch further comprises an active.

10. A device according to claim 9, wherein the active is a drug.

11. A device according to claim 10, wherein the tab of the release substrate is completely laterally offset from the combination.

12. A kit containing an application device comprising:
(a) an application device according to claim 6;
(b) a usage instructions associated therewith;
(c) a package containing components (a) and (b).
